(19) United States
(12) Patent Application Publication
(10) Pub. No.: US 2026/0134974 A1
(43) Pub. Date: May 14, 2026

(54) SYSTEMS AND METHODS FOR EVALUATING BEHAVIORAL DISORDERS, DEVELOPMENTAL DELAYS, AND NEUROLOGIC IMPAIRMENTS

(71) Applicant: Cognoa, Inc., Palo Alto, CA (US)

(72) Inventors: Dennis WALL, Palo Alto, CA (US); Stuart Angus LIU-MAYO, Palo Alto, CA (US)

(21) Appl. No.: 19/120,700

(22) PCT Filed: Oct. 16, 2023
(86) PCT No.: PCT/US2023/077015

§ 371 Date: Apr. 11, 2025

Related U.S. Application Data
(60) Provisional application No. 63/416,420, filed on Oct. 14, 2022.
(60) Provisional application No. 63/416,422, filed on Oct. 14, 2022.

(51) Int. Cl. G16H 20/70; G16H 20/10; G16H 50/20; G16H 50/70

(52) U.S. Cl. CPC G16H 20/70; G16H 20/10; G16H 50/20; G16H 50/70

(57) ABSTRACT

Described herein are systems and methods used to evaluate individuals such as children for behavioral disorders, developmental delays, and neurological impairments. An exemplary method includes receiving input data of an individual related to a behavioral disorder, neurological impairment, or developmental delay, and evaluating the input data using an evaluation module comprising at least one machine learning model, thereby generating an evaluation result, where the machine learning model comprises one or more decision threshold hyperparameters that differentiate between a positive evaluation, negative evaluation, and an indeterminate evaluation with respect to a presence or an absence of the behavioral disorder, neurological impairment, or developmental delay.

### CROSS-REFERENCE

This application claims the benefit of U.S. Patent Application No. 63/416,420, filed Oct. 14, 2022, and U.S. Patent Application No. 63/416,422, filed Oct. 14, 2022, each of which is incorporated by reference herein in its entirety.

### BACKGROUND

Numerous individuals including children suffer from behavioral disorders, developmental delays, and neurological impairments. Examples of these conditions include attention deficit hyperactivity disorder (ADHD), autism (e.g., autism spectrum disorder, ASD), and speech disorders.

Healthcare providers typically evaluate behavioral disorders, developmental delays, and neurological impairments using traditional observational techniques such as questionnaires and clinician interviews.

### SUMMARY

Described herein are methods, devices, systems, software, and platforms used to evaluate individuals such as children for behavioral disorders, developmental delays, and neurological impairments. Also described herein are methods, devices, systems, software, and platforms that are used to increase the accuracy and efficiency of evaluating individuals having one or more behavioral disorders, developmental delays, and neurological impairments. As compared to traditional techniques, the methods, devices, systems, software, and platforms described herein can utilize input data associated with an individual and generate a diagnosis thereof with high accuracy. The inventive methods, devices, systems, software, and platforms described herein are designed at least in part to provide treatment to individuals who suffer from behavioral disorders, developmental delays, and neurological impairments.

Evaluating behavioral disorders, developmental delays, and neurological impairments accurately and efficiently can be challenging. A potential reason is that these health conditions often have overlapping symptoms, which make it difficult to distinguish from one another. For example, one type of developmental delay such as autism, has overlapping symptoms with another type of developmental delay such as speech delay. Another example is that behavioral disorder (e.g., ADHD) has overlapping symptoms with developmental delay (e.g., autism). Hence, patients often receive an incorrect diagnosis or an incomplete one, where patients with multiple health conditions only have one condition diagnosed.

Questionnaires and clinician interviews are often used to diagnose patients by collecting multiple types of data including a variety of test findings. Both questionnaires and clinician interviews involve long question sets that are administered to patients and/or respective caretakers, which can be costly and inefficient in terms of time and resources. Questionnaire data can be of limited value, as the quality of the data highly depends on the subject's attention span and willingness to participate. Therefore, questionnaire data can be incomplete or inaccurate.

Another challenge in diagnosing patients, especially younger patients suffering from behavioral disorders, developmental delays, and neurological impairments, is timing. While reliable autism diagnosis is possible as early as 18 months, the average age of diagnosis remains above four years in the United States. Multiple factors contribute to diagnostic delays, including structural drivers of inequity such as poverty, racism and gender bias. While a number of innovative physician training programs have shown that diagnosing health issues (e.g., autism) in younger patients in primary care settings is feasible, many primary care providers still report they are under-equipped and/or under-staffed. Existing diagnostic tools for autism, despite reasonable accuracy and inter-rater reliability, are often difficult to use. These tools may take a long time to administer, may not be amenable to telemedicine, and often require specialist training.

In contrast, described herein are methods, devices, systems, software, and platforms for accurately and efficiently assessing individuals for at least one condition type selected from multiple conditions including behavioral disorders, developmental delays, and/or neurological impairments. Also described herein are methods, devices, systems, software, and platforms to evaluate individuals using a trained model having one or more decision threshold hyperparameters that provide improved inclusion, accuracy, and/or other performance metrics. The output of the trained model comprises a positive evaluation, a negative evaluation, or an indeterminate evaluation.

In some aspects, disclosed herein is a computer-implemented method comprising: (a) receiving input data of an individual related to a behavioral disorder, neurological impairment, or developmental delay; (b) evaluating the input data using an evaluation module comprising at least one machine learning model, thereby generating an evaluation result, wherein the at least one machine learning model comprises one or more decision threshold hyperparameters that differentiate between a positive evaluation, negative evaluation, and an indeterminate evaluation with respect to presence or absence of the behavioral disorder, neurological impairment, or developmental delay; and (c) generating a personal therapeutic treatment plan for the individual based at least in part on the evaluation result if the evaluation result comprises the presence of the behavioral disorder, neurological impairment, or developmental delay.

In some embodiments, the at least one machine learning model comprising the one or more decision threshold hyperparameters provides a positive predictive value of at least about 80%, a negative predictive value of at least about 95%, a coverage or inclusion rate of at least about 70%, or any combination thereof when evaluated using a nested cross-validation procedure. In some embodiments, the one or more decision threshold hyperparameters are generated using an automated cross-validation procedure. In some embodiments, the one or more decision threshold hyperparameters define a threshold range for determining if an evaluation result is a positive evaluation, a negative evaluation, or an indeterminate evaluation.

In some embodiments, the first categorical determination for the presence or absence of the behavioral disorder, neurological impairment, or developmental delay in the individual is based on a specified sensitivity, a specified specificity, a specified negative predictive value, or a specified positive predictive value.

In some embodiments, the at least one machine learning model comprises a plurality of tunable machine learning models.

In some embodiments, the method further comprises: (a) requesting additional data if the evaluation result comprises the indeterminate evaluation; and (b) generating an updated evaluation result based on the additional data using the evaluation module. In some embodiments, the method further comprises: (a) combining scores for each of a plurality of tunable machine learning models to generate a combined preliminary output score; and (b) comparing the combined preliminary output score to the one or more decision threshold hyperparameters to generate an updated evaluation result. In some embodiments, the combined preliminary output score is based on a rule-based logic or a combinatorial technique for combining the scores. In some embodiments, the method further comprises training the at least one machine learning model with a first training dataset, a second training dataset, and a third training dataset, wherein the first training dataset comprises one or more video recordings of individuals, the second training dataset comprises one or more feedbacks provided by healthcare providers to a questionnaire, and the third training dataset comprises one or more feedbacks provided by caregivers to a questionnaire. In some embodiments, at least one of the first training dataset, the second training dataset, or the third training dataset comprises at least 100 training samples.

In some embodiments, the behavioral disorder, neurological impairment, or developmental delay comprises pervasive development disorder (PDD), autism spectrum disorder (ASD), social communication disorder, restricted repetitive behaviors, interests, and activities (RRBs), autism (e.g., classical autism), Asperger's Syndrome (e.g., high functioning autism), PDD-not otherwise specified (PDD-NOS, atypical autism), attention deficit disorder (ADD), attention deficit and hyperactivity disorder (ADHD), speech and language delay, obsessive compulsive disorder (OCD), depression, schizophrenia, Alzheimer's disease, dementia, intellectual disability, or learning disability. In some embodiments, the behavioral disorder, neurological impairment, or developmental delay is autism spectrum disorder or autism.

In some embodiments, the method further comprises generating a personal therapeutic treatment plan for the individual based on the evaluation result. In some embodiments, the personal therapeutic treatment plan is generated using a therapeutic module comprising at least one statistical or machine learning model. In some embodiments, the method further comprises receiving feedback data based on performance of the personal therapeutic treatment plan and updating the personal therapeutic treatment plan based on the feedback data. In some embodiments, the feedback data comprises at least one of efficacy, compliance, and response to the personal therapeutic treatment plan. In some embodiments, the personal therapeutic treatment plan comprises a drug therapy, a non-drug therapy, or both. In some embodiments, the non-drug therapy comprises digital therapeutics. In some embodiments, the drug therapy comprises use of one or more drugs to treatment the behavioral disorder, neurological impairment, or developmental delay. The therapeutic module generates recommendation of one or more drugs, and respective dosage and timing to the individual, based at least in part on the evaluation result of the individual. For example, for an individual with a positive evaluation of behavioral disorder, the therapeutic module generates recommendation of drug therapy including one or more of stimulants (e.g., mixed amphetamine salts, methylphenidate), nonstimulant ADHD medicines (e.g., atomoxetine, guanfacine ER), anticonvulsant medicine (e.g., divalproex), antipsychotics (e.g., aripiprazole, risperidone, ziprasidone). In some embodiments, the non-drug therapy comprises cognitive therapy, behavioral therapy, occupational therapy (e.g., sensational integration therapy), physical therapy, speech and language therapy, lifestyle change, physiotherapy, and pain management. For example, behavioral therapy may include discrete trial training (DTT) which uses step-by-step instructions to teach a desired behavior or response. Lessons are broken down into their simplest parts, and desired answers and behaviors are rewarded. Undesired answers and behaviors are ignored. As another example, or in conjunction with DTT, pivotal response training (PRT) may be used, which takes place in a natural setting rather than clinic setting. The goal of PRT is to improve a few “pivotal skills” that will help the person learn many other skills. One example of a pivotal skill is to initiate communication with others.

In some embodiments, the digital therapeutics comprises a single or multiplicity of therapeutic activities or interventions that can be performed by the individual and/or respective caregiver/healthcare provider. The digital therapeutics includes prescribed interactions with third party devices including sensors, computers, medical devices and therapeutic delivery systems. The digital therapeutics can support an FDA approved medical claim, a set of diagnostic codes, a single diagnostic code. In some other embodiments, the digital therapeutics comprises instructions, feedback, activities or interactions provided to the individual and/or respective caregiver/healthcare provider. Examples include suggested behaviors, activities, games or interactive sessions with third party devices (e.g., the Internet of Things “IoT” enabled therapeutic devices). Additional descriptions and examples for digital therapeutics are included in PCT Application No. PCT/US2018/017354, which is incorporated by reference in its entirety for all purposes.

In some embodiments, the therapeutic module comprises a recommendation engine that provides a personal treatment plan based at least in on the evaluation result. The recommendation engine receives the evaluation result generated from the evaluation module that an individual has a behavioral disorder, and generates the personal treatment plan. The personal treatment plan comprises a therapeutic treatment plan, including but is not limited to, recommendations of courses of actions, therapies for individuals to be engaged in, and daily activities for individuals to be engaged in.

In other embodiments, in addition to the evaluation result, the recommendation engine receives data associated with the individual to generate the personal treatment plan. The additional data about the individual includes but is not limited to, demographical data, metabolic data, pharmacokinetic data, clearance data, and microbiome data. Demographical data comprises an individual's age, sex, height, weight, diagnostic status for one or more disorders, and/or any other relevant demographic data. Metabolic data can be relevant to assessing the efficacy of the use of a therapy to treat a behavioral disorder. Metabolic data comprises measurements of one or more metabolites (e.g., creatinine, xanthine, hypoxanthine, inosine) associated with the use of a therapy to treat a behavioral disorder. The pharmacokinetics of an individual can be determined in response to administering a known amount of the therapeutic agent to the individual at a first time and determining an amount of the therapeutic agent at a second time. For example, the known amount of the therapeutic agent can be administered to the individual at the beginning of the therapy, while the physiological parameters and metabolic data of the individual are monitored. When the physiological parameters and metabolic data indicate the severe side effects of the therapeutic agent, a lower dosage and/or less frequent timing can be recommended for a later time during the therapy. Measured pharmacokinetic data can be selected from the group consisting of an alpha elimination half-life and a beta elimination half-life. The clearance data of an individual comprises clearance rate of a therapeutic agent in the body of the individual. The microbiome data of an individual comprises data selected from a stool sample, intestinal lavage, or other sample of the flora of the individual's intestinal track.

In some embodiments, the recommendation engine receives the evaluation result generated from the evaluation module that an individual has a behavioral disorder, and generates a recommendation of one or more types of drugs to the individual. In other embodiments, the recommendation engine generates a recommendation of the timing and/or dosage of the drug.

In some embodiments, the method further comprises administering a treatment to the individual if the evaluation result comprises the presence of the behavioral disorder, neurological impairment, or developmental delay. The treatment can be selected from the group consisting of psychotherapy, behavioral treatment, pharmacologic therapy, and surgical intervention.

In some aspects, disclosed herein is a computer-implemented method comprising: (a) receiving input data comprising a plurality of features related to a behavioral disorder, neurological impairment, or developmental delay; (b) dividing the input data into training data sets and testing data sets; (c) training a model using one of the training data sets; (d) determining at least one decision threshold(s) for the model using a corresponding testing data set; (e) repeating steps (b)-(d) at least once using a cross-validation procedure to generate a plurality of decision thresholds; (f) determining one or more decision threshold hyperparameters using the plurality of decision thresholds; and (g) training a final model using the input data, wherein the final model comprises the one or more decision threshold hyperparameters.

In some aspects, disclosed herein is a computer-implemented method comprising: (a) receiving input data comprising a plurality of features related to a behavioral disorder, neurological impairment, or developmental delay; (b) dividing the input data into tuning data sets and validation data sets; (c) dividing the tuning data sets into training data sets and testing data sets; (d) training a model using one of the training data sets; (e) determining at least one decision threshold(s) for the model using a corresponding testing data set; (f) repeating steps (c)-(e) at least once using a cross-validation procedure to generate a plurality of decision thresholds; (g) training a new model using one of the tuning data sets; (h) evaluating the new model using a corresponding validation data set to generate at least one decision threshold based on the plurality of decision thresholds; (i) repeating steps (a)-(h) at least once using a cross-validation procedure to calculate one or more performance metrics, (j) determining one or more decision threshold hyperparameters using the plurality of decision thresholds; and (k) training a final model using the input data, wherein the final model comprises the one or more decision threshold hyperparameters.

In some aspects, disclosed herein is a computer-implemented method comprising: (a) receiving input data of an individual related to a behavioral disorder, neurological impairment, or developmental delay; (b) evaluating the input data using a machine learning model to generate an evaluation result comprising a positive evaluation, negative evaluation, and an indeterminate evaluation with respect to presence or absence of the behavioral disorder, neurological impairment, or developmental delay; and (c) generating a personal therapeutic treatment plan for based at least in part on the evaluation result if the evaluation result comprises the presence of the behavioral disorder, neurological impairment, or developmental delay.

In some aspects, disclosed herein is a system comprising: a processor; a non-transitory computer readable medium including executable instructions configured to cause the processor to perform the computer-implemented method of any one of the disclosed embodiments.

In some aspects, disclosed herein is a non-transitory computer readable medium including executable instructions configured to cause a processor to perform the computer-implemented method of any one of the disclosed embodiments.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a workflow of evaluating individuals for the presence of Autism Spectrum Disorder (ASD) using caregiver input by answering a questionnaire, video input of the individual in home setting, and health care provider input by answering a questionnaire.

FIG. 2 shows a diagram of the lower and upper thresholds delineating the cutoffs for making predictions of absence of ASD and presence of ASD, respectively. Any score between the lower and upper threshold results in an abstention.

FIG. 3 shows a chart of the neurodevelopmental composition of children in the combined dataset.

FIG. 4 shows a map indicating the children from 43 states were represented in the combined dataset.

FIG. 5A and FIG. 5B provide an illustration of the effect of the model's threshold hyperparameters on performance metrics, where the algorithm (“algorithm V2”) had a 66.5% determinate rate following optimization.

FIG. 6 shows the Canvas DX™ performance metrics with algorithm V2.

FIG. 7A shows abstention rates and neurodevelopmental composition using algorithm V1.

FIG. 7B shows abstention rates and neurodevelopmental composition using algorithm V2.

FIG. 8 illustrates a computer system that is programmed or otherwise configured to implement methods provided herein.

### DETAILED DESCRIPTION

Described herein are systems and computer-implemented methods used to evaluate individuals including children for behavioral disorders, developmental delays, and neurological impairments. Computer-implemented methods described herein are configured in various embodiments to be run on one or more computing devices, within one or more computing system, or on one or more platforms.

In some aspects, disclosed herein is a computer-implemented method comprising: (a) receiving input data of an individual related to a behavioral disorder, neurological impairment, or developmental delay; (b) evaluating the input data using an evaluation module comprising at least one machine learning model, thereby generating an evaluation result, wherein the at least one machine learning model comprises one or more decision threshold hyperparameters that differentiate between a positive evaluation, negative evaluation, and an indeterminate evaluation with respect to a presence or an absence of the behavioral disorder, neurological impairment, or developmental delay.

The input data of an individual related to a behavioral disorder, neurological impairment, or developmental delay may be collected from the individual directly or from respective caretakers and/or healthcare providers. In some embodiments, the input data may comprise one or more video recordings of an individual, in which the individual may be performing tasks and/or interacting with others. In some other embodiments, trained analysts may review the video recordings, observe behaviors of the individual, and answer a questionnaire. The questionnaire comprises about 5-50 questions, for example, “How often does the child try to get people's attention?” “How would you describe the child's activity level?” The video recordings of the individual and/or answers to the questionnaire provided by the analysts may be used as the input data for the evaluation module to process. In other embodiments, caretakers and/or healthcare providers may also answer one or more questionnaires, which may be used as input data to the evaluation module.

In some embodiments, the method may comprise training the at least one machine learning model with a first training dataset (e.g., input data comprising feedback from a caretaker), a second training dataset (e.g., input data comprising evaluation of a video recording), and a third training dataset (e.g., input data comprising feedback from a healthcare provider), wherein the first training dataset comprises one or more video recordings of individuals, the second training dataset comprises one or more feedbacks provided by healthcare providers to a questionnaire, and the third training dataset comprises one or more feedbacks provided by caregivers to a questionnaire. In some embodiments, at least one of the first training dataset, the second training dataset, or the third training dataset comprises at least 100 training samples.

The questionnaires may comprise a variety of questions targeted to evaluate the development, behavior, and/or other attributes of the individual. The number and/or type of questions in the questionnaire may be different, depending on the age of the individual (e.g., 18-47 months, 48-72 months), and the role of the person who answers the questionnaire (e.g., caregiver, health care provider, analyst reviewing the video recording of the individual). In some embodiments, a questionnaire targeted to a healthcare provider of a 18-47 month child may comprise about 5-30 questions, for example, “does the child have repetitive whole body movement?” and “how is the child's level of eye contact?” A questionnaire targeted to a healthcare provider of a 48-72 month child may comprise about 5-30 questions, for example, “does the child spontaneously imitate parents or other people in the family?” In some other embodiments, a questionnaire targeted to a caregiver of a 18-47 month child may comprise about 5-30 questions, for example, “does your child typically share his/her excitement or enjoyment with you or others? Consider when he/she is excited about a new toy or about going somewhere, or anything else that gets him/her excited.” A questionnaire targeted to a caregiver of a 48-72 month child may comprise about 5-30 questions, for example, “does your child try to comfort others without being told to or without any sort of prompting?” In some other embodiments, a questionnaire targeted to an analyst reviewing a video recording of a 18-47 month child may comprise about 5-50 questions, for example, “does the child exhibit any self-harming behaviors?” A questionnaire targeted to an analyst reviewing a video recording of a 48-72 month child may comprise about 5-50 questions, for example, “how would you describe the child's use of gestures?”

In some embodiments, the questionnaire may provide categorical answer choices. For example, for a question “How would you describe the child's vocalizations toward others?” The questionnaire may provide a variety of answer choices including “Excellent: Numerous examples of social communication or vocalizations that clearly express interest or make needs known” which may correspond to a score of 0, “Good: Examples of social communication are sometimes observed and clearly express interest and make needs known” which may correspond to a score of 1, “Satisfactory: An example of social communication is observed that clearly expresses interest or makes a need known” which may correspond to a score of 2, “Poor: No examples of social communication are observed or the child does not vocalize” which may correspond to a score of 3, and “The footage doesn't provide sufficient opportunity to assess reliably” which may correspond to a score of 9. These answers, or the scores corresponding to the answers, may be used by the evaluation module to calculate the evaluation result of the individual. In some embodiments, the scores may be used to train the machine learning model. In some embodiments, the evaluation module may rank or weigh the questions and/or answers provided, depending on the age of the individual, the type of behavioral disorder, neurological impairment, or developmental delay to be diagnosed, and/or other factors related to the diagnosis of the individual.

In some embodiments, the questionnaire may include a pre-determined or a set number of questions. For example, the evaluation module may generate a questionnaire customized to the individual's condition. The evaluation module may take into account the demographic information (e.g., age, gender) and current medical condition of the individual, and generate an individualized questionnaire having a predetermined or a set number of questions. In other embodiments, the evaluation module may dynamically adjust the number and/or type of questions in the questionnaire based on the answers provided by caregivers, healthcare providers, and/or video analysts to previous questions. For example, if the answers to questions related to developmental delay show significant under development of the individual, the evaluation module may include additional questions that are targeted to developmental delay evaluation. In some embodiments, the evaluation module may comprise one or more natural language processing models that are configured to generate questions targeted to the evaluation of the individual and process free form answers provided by caretakers and healthcare providers. The natural language processing model may provide more flexibility and accuracy in the questionnaires and answers provided, thereby facilitating the diagnosis and treatment of the individual.

In some embodiments, the at least one machine learning model comprising the one or more decision threshold hyperparameters provides a positive predictive value of at least about 80%, a negative predictive value of at least about 95%, a coverage or inclusion rate of at least about 70%, or any combination thereof when evaluated using a nested cross-validation procedure. The use of one or more decision threshold hyperparameters can yield improved performance compared to the scenario where multiple models are used for prediction, and each model can only process input data collected from individuals within a narrower range of age group. In some embodiments, the positive predictive value is at least about 70%, about 75%, about 80%, about 85%, about 90%, about 95%, or about 99%. In some embodiments, the negative predictive value is at least about 70%, about 75%, about 80%, about 85%, about 90%, about 95%, or about 99%. In some embodiments, the coverage or inclusion rate (e.g., evaluation results after subtracting indeterminate results) is at least about 50%, about 55%, about 60%, about 65%, about 70%, about 75%, about 80%, about 85%, about 90%, about 95%, or about 99%.

A variety of procedures can be used to select decision thresholds. For example, decision thresholds can be determined based on feasibility studies, including the distribution of numeric output generated by the machine learning models, the response distributions for the questions on the questionnaires, and the sample metadata and covariates. Clinical assumptions can also be incorporated about the likely composition of the target population in determining the decision thresholds.

Disclosed herein are systems and methods that utilize a novel decision threshold selection process that produces robust performance estimates for future usage of the model, mitigates overfitting risk, and minimizes the impact of human biases in the loop. The use of the systems and methods as described herein by healthcare providers does not require specialist training and can occur remotely or in-person.

In some embodiments, the systems and methods utilize machine learning to assist healthcare providers to diagnose or rule out behavioral disorder, neurological impairment, or developmental delay (e.g., autism) in children aged 18 through 72 months. The systems and methods as described herein can collect and integrate data from caregivers and healthcare providers, together with structured observations of the child based on videos recorded at the home setting. The machine learning model can be trained to produce one of three outputs: positive, negative, or indeterminate for behavioral disorder, neurological impairment, or developmental delay (e.g., autism). The indeterminate category may be a safeguard that enables abstention if presented with insufficient data for determinate classification. Abstention in cases of high uncertainty is a valuable machine learning-based medical device to safeguard and minimize the risk of false clinical outcomes. The machine learning model can be configured for iterative learning and performance improvements with exposure to additional real-world data. Efficiently learning new tasks and incorporating new training data, sometimes referred to as “life-long learning,” presents a number of challenges. Training data selection, for example, needs to be appropriate to the clinical problem of interest and represent the diverse phenotypes of the intended population to avoid amplification of gender, racial, socio-economic or other demographic biases. Obtaining reliable labels for conditions without objective diagnostic tests can present another challenge, as can implementation of procedures for robust model validation against covariates of relevance.

The interest in the potential of both supervised and unsupervised machine learning approaches to prediction of behavioral disorder, neurological impairment, or developmental delay and respective treatment delivery tasks continues to grow. Artificial intelligence/machine learning software as a medical device (SaMD) is often required to be “locked” at the point of regulatory approval, or conducting a full clinical trial prior to any contemplated changes. The locking of the SaMD may bring risks that hinder SaMD from rapidly evolving and enhancing performance over time with exposure to new data.

The Predetermined Change Control Plan (PCCP) was proposed to regulate artificial intelligence/machine learning software as a medical device by Food and Drug Administration (FDA) in 2019. PCCP outlines a process by which approved algorithms can be intermittently “unlocked” under certain circumstances and with specific guardrails in place, in order to leverage new data to enhance performance or address issues of concern. Both types of anticipated software modifications (e.g., SaMD pre-specifications) and approaches to making changes so that devices remain safe and effective following algorithmic modifications (e.g., algorithm change protocols) are considered. Non-adaptive artificial intelligence/machine learning-based algorithms risk becoming dated and degraded over time if data used for training no longer reflects the real-world circumstances that they are being applied to. By providing a mechanism that allows manufacturers to intermittently “unlock” algorithms to expose their models to additional data, PCCPs may help to prevent algorithmic drift.

In some embodiments, the systems and methods as described herein use abstention thresholds that are modified under the PCCP. The modification increases the determinate rate of the machine learning model without changing its intended use and while maintaining the reliability in the predictions. This novel real world threshold update conducted under the PCCP fills the research gap regarding best practice regulation of SaMD with the potential for iterative learning if exposed to new data.

In some embodiments, the systems and methods as described herein include a nested cross-validation procedure in which the following steps are performed:
1. Split input data into tuning data sets and validation data sets.
2. Split the tuning data sets into training data sets and testing data sets.
3. Train the model on one of the training data sets.
4. Identify the optimal decision thresholds using a corresponding testing data set.
5. Repeat steps 2-4 via cross-validation.
6. Retrain the model on the tuning data sets—training on full tuning data sets—no longer splitting into subsets.
7. Using the mean of the decision thresholds identified in the inner c-v loop, evaluate the model trained in step 6 on the validation data set.
8. Repeat steps 1-7 via cross-validation.

In some embodiments, the tuning data may be randomly divided with, for example, 90% as training data sets and 10% as testing data sets, 80% as training data sets and 20% as testing data sets, 70% as training data sets and 50% as testing data sets, 60% as training data sets and 40% as testing data sets, 50% as training data sets and 50% as testing data sets, 40% as training data sets and 60% as testing data sets. The tuning data may have ground truth labels. The machine learning model may be trained using part of the training data sets and generate one or more decision thresholds using a corresponding testing data set. For example, the prediction results generated by the machine learning model may be compared with ground truth labels, which determines whether the decision thresholds are optimal. These steps may be repeated iteratively and during which, the tunning data may be shuffled, redivided into training data sets and testing data sets, and used by the machine learning model to generate prediction results.

In some embodiments, this procedure may produce quantifiable performance estimates that ensure the machine learning model can function as designed if used in a real-life setting. Afterwards, a final training procedure may be optionally performed to further fine-tune the model:
9. Split input data into training data sets and testing data sets that may be different from the original training and testing data sets.
10. Train the model on one of the training data sets.
11. Identify the optimal decision thresholds using a corresponding testing data set.
12. Repeat steps 9-11 via cross validation.
13. Determine the final decision thresholds as the mean of the decision thresholds identified in the step 12 loop.
14. Train a final model on the full data set.

In some embodiments, if the full nested cross-validation procedure is performed, it results a trained machine learning model with one or more optimized decision threshold hyperparameters (e.g., thresholds delineating between positive and indeterminate evaluations, as well as between indeterminate and negative evaluations) for use in a computing device or program for evaluating an individual for a behavioral disorder, developmental delay, or neurological impairment. In addition, the nested cross-validation technique provides robust, trustworthy estimates of the model's real-world performance.

The evaluation system, device, and methods as described herein can classify at least one behavioral disorder, developmental delay, or neurological impairment with improved sensitivity and specificity. Additionally, the evaluation system, device, and methods are able to be continuously improved as more data from the target population becomes available for use in the model-building process.

In some embodiments, an individual is evaluated by a series of prompts in the form of questions displayed on a screen of a computing device.

In some embodiments, an individual is evaluated by recorded video and/or audio data of the individual interacting with other people, carrying out tasks, and/or answering questions. In some embodiments, the individual is recorded answering questions asked by a human questioner or caretaker. In some embodiments, a video analyst answers a questionnaire based on one or more recorded videos of the individual.

In some embodiments, a questionnaire is completed by individuals, or their respective caretakers, or health care providers on a mobile device or stationary computing device. In some embodiments, a video and/or audio recording is taken with a mobile device. In some embodiments, the mobile device is a smartphone, a tablet, a smartwatch, or any device with a mobile camera or recording feature. In some embodiments, the video and/or audio recording is taken with a stationary camera and/or microphone. For example, an individual can be asked questions in a clinician's office and have their responses recorded with a camera on a tripod with a mounted microphone.

Some non-limiting examples of conditions classified as behavioral disorders comprise Attention Deficit Hyperactivity Disorder (ADHD), Oppositional Defiant Disorder (ODD), Autism Spectrum Disorder (ASD), Anxiety Disorders, Depression, Bipolar Disorders, Learning Disorders or Disabilities, or Conduct Disorder. In some embodiments, an Attention Deficit Hyperactivity Disorder (ADHD) comprises Predominantly Inattentive ADHD, Predominantly Hyperactive-impulsive type ADHD, or Combined Hyperactive-impulsive and Inattentive type ADHD. In some embodiments, Autism Spectrum Disorder (ASD) comprises Autistic Disorder (classic autism), Asperger Syndrome, Pervasive Developmental Disorder (atypical autism), or Childhood disintegrative disorder. In some embodiments, Anxiety Disorders comprise Panic Disorder, Phobia, Social Anxiety Disorder, Obsessive-Compulsive Disorder, Separation Anxiety Disorder, Illness Anxiety Disorder (Hypochondria), or Post-Traumatic Stress Disorder. In some embodiments, Depression comprises Major Depression, Persistent Depressive Disorder, Bipolar Disorder, Seasonal Affective Disorder, Psychotic Depression, Peripartum (Postpartum) Depression, Premenstrual Dysphoric Disorder, “Situational” Depression, or Atypical Depression. In some embodiments, Bipolar Disorders comprise Bipolar I Disorder, Bipolar II Disorder, Cyclothymic Disorder or Bipolar Disorder due to another medical or substance abuse disorder. In some embodiments, Learning Disorders comprise Dyslexia, Dyscalculia, Dysgraphia, Dyspraxia (Sensory Integration Disorder), Dysphasia/Aphasia, Auditory Processing Disorder, or Visual Processing Disorder. In some embodiments, Behavioral Disorder is a disorder defined in any edition of the Diagnostic and Statistical Manual of Mental Disorders (DSM).

Some non-limiting examples of conditions classified as developmental delays comprise Autism Spectrum Disorder, Mental Retardation, Cerebral Palsy, Down Syndrome, Failure to Thrive, Muscular Dystrophy, Hydrocephalus, Developmental Coordination Disorder, Cystic Fibrosis, Fetal Alcohol Syndrome, Homocystinuria, Tuberous Sclerosis, Abetalipoproteinemia, Phenylketonuria, Aase Syndrome, speech delays, gross motor delays, fine motor delays, social delays, emotional delays, behavioral delays, or cognitive delays. In some embodiments, Mental Retardation comprises Adrenoleukodystrophy, Ito Syndrome, Acrodysostosis, Huntington's Disease, Aarskog Syndrome, Aicardi Syndrome or Tay-Sachs Disease. In some embodiments, Cerebral Palsy comprises Spastic Cerebral Palsy, Dyskinetic Cerebral Palsy, Hypotonic Cerebral Palsy, Ataxic Cerebral Palsy, or Mixed Cerebral Palsy. In some embodiments, Autism Spectrum Disorder comprises Autistic Disorder (classic autism), Asperger Syndrome, Pervasive Developmental Disorder (atypical autism), or Childhood disintegrative disorder. In some embodiments, Down Syndrome comprises Trisomy 21, Mosaicism, or Translocation. In some embodiments, Muscular Dystrophy comprises Duchenne muscular dystrophy, Becker muscular dystrophy, Congenital muscular dystrophy, Myotonic dystrophy, Facioscapulohumeral muscular dystrophy, Oculopharyngeal muscular dystrophy, Distal muscular dystrophy, or Emery-Dreifuss muscular dystrophy.

Some non-limiting examples of conditions classified as neurological impairments comprise Amyotrophic Lateral Sclerosis, Arteriovenous Malformation, brain aneurysm, brain tumors, Dural Arteriovenous Fistulae, Epilepsy, headache, memory disorders, Multiple Sclerosis, Parkinson's Disease, Peripheral Neuropathy, Post-Herpetic Neuralgia, spinal cord tumor, stroke, Alzheimer's Disease, Corticobasal Degeneration, Creutzfeldt-Jakob Disease, Frontotemporal Dementia, Lewy Body Dementia, Mild Cognitive Impairment, Progressive Supranuclear Palsy, or Vascular Dementia.

In some embodiments, the methods described herein are used in conjunction with known techniques of diagnosing behavioral disorders, developmental delays, or neurological impairments. In some embodiments, the methods disclosed herein are used to aid in the diagnosis behavioral disorders, developmental delays, or neurological impairments. In some embodiments, the methods described herein can enhance the accuracy of known methods of diagnosis, or reduce the time or recourses required for accurate diagnosis.

The software described herein, in some embodiments, is located in a computing device used to receive the input to the software. In some embodiments, software as described herein is located on a server that is communicatively coupled with a computing device used by the individual being evaluated.

### Machine Learning Software Modules

As described above, in some embodiments of the methods, devices, systems, software, and platforms described herein, an evaluation module is utilized to evaluate input data for an individual and generate an evaluation with respect to at least one behavioral disorder, developmental delay, or neurological impairment. In some embodiments, the evaluation module comprises one or more trained models or machine learning algorithms that process the individual's input data and generate an output that is indicative of a positive, negative, or indeterminate evaluation of the at least one behavioral disorder, developmental delay, or neurological impairment. In some other embodiments of the methods, devices, systems, software, and platforms described herein, a therapeutic module is utilized to generate a personal therapeutic treatment plan for the individual based on the evaluation result. The therapeutic module comprises at least one statistical or machine learning model.

A given trained machine learning model can have one or more decision threshold hyperparameters that delineate the boundaries between the positive, negative, and indeterminate outputs. For example, an output generated by the model can be a score that is compared against decision threshold hyperparameters to determine the final evaluation outcome. It should be understood that machine learning encompasses numerous architectures and arrangements of data and that the teachings herein are not limited to any one single type of machine learning.

A machine learning model described herein can be trained using datasets from individuals with a known positive or negative diagnosis of one or more of behavioral disorders, developmental delays, or neurological impairments. The individuals may have been previously evaluated for symptoms of the one or more of behavioral disorders, developmental delays, or neurological impairments. In some embodiments, input data may be divided into training data sets and testing data sets. The machine learning model may be trained using one of the training data sets, which may determine at least one decision threshold for the model using a corresponding testing data set. The division of input data, training the machine learning model, and determining the at least one decision threshold may be repeated using a cross-validation procedure.

In some embodiments, the machine learning model comprises one or more supervised, semi-supervised, self-supervised, or unsupervised machine learning techniques. For example, a machine learning model may be a trained model that is trained through supervised learning (e.g., various parameters are determined as weights or scaling factors).

Training the machine learning model may include, in some cases, selecting one or more untrained data models to train using a training data set. The selected untrained data models may include any type of untrained machine learning models for supervised, semi-supervised, self-supervised, or unsupervised machine learning. The selected untrained data models be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the machine learning model from the selected untrained data models may likewise be selected, such as limits on the machine learning model complexity or limits on the machine learning model refinement past a certain point. The machine learning model may be trained (e.g., via a computer system such as a server) using the training data set. In some cases, a first subset of the training data set may be selected to train the machine learning model. The selected untrained data models may then be trained on the first subset of training data set using appropriate machine learning techniques, based upon the type of machine learning model selected and any conditions specified for training the machine learning model. In some cases, due to the processing power requirements of training the machine learning model, the selected untrained data models may be trained using additional computing resources (e.g., cloud computing resources). Such training may continue, in some cases, until at least one aspect of the machine learning model is validated and meets selection criteria to be used as a predictive model.

In some cases, one or more aspects of the machine learning model may be validated using a second subset of the training data set (e.g., distinct from the first subset of the training data set) to determine accuracy and robustness of the machine learning model. Such validation may include applying the machine learning model to the second subset of the training data set to make predictions derived from the second subset of the training data. The machine learning model may then be evaluated to determine whether performance is sufficient based upon the derived predictions. The sufficiency criteria applied to the machine learning model may vary depending upon the size of the training data set available for training, the performance of previous iterations of trained models, or user-specified performance requirements. If the machine learning model does not achieve sufficient performance, additional training may be performed. Additional training may include refinement of the machine learning model or retraining on a different first subset of the training dataset, after which the new machine learning model may again be validated and assessed. If the machine learning model has achieved sufficient performance, in some cases, the machine learning may be stored for present or future use. The machine learning model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of machine learning models may be stored for generating predictions under different sets of input data conditions. In some embodiments, the machine learning model may be stored in a database (e.g., associated with server).

The machine learning model may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, alternating decision trees, best-first decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

In some embodiments, the machine learning model may implement a decision tree. A decision tree may be a supervised machine learning algorithm that can be applied to both regression and classification problems. Decision trees may mimic the decision-making process of a human brain. For example, a decision tree may grow from a root (base condition), and if it meets a condition (internal node/feature), it may split into multiple branches. The end of the branch that does not split anymore may be an outcome (leaf). A decision tree can be generated using a training data set according to the following operations: (1) starting from a root node (the entire dataset), the algorithm may split the dataset in two branches using a decision rule or branching criterion; (2) each of these two branches may generate a new child node; (3) for each new child node, the branching process may be repeated until the dataset cannot be split any further; (4) each branching criterion may be chosen to maximize information gain (e.g., a quantification of how much a branching criterion reduces a quantification of how mixed the labels are in the children nodes). The labels may be the data or the classification that is predicted by the decision tree.

A random forest regression is an extension of the decision tree model that tends to yield more robust predictions by stretching the use of the training data partition. Whereas a decision tree may make a single pass through the data, a random forest regression may bootstrap 50% of the data (e.g., with replacement) and build many trees. Rather than using all explanatory variables as candidates for splitting, a random subset of candidate variables may be used for splitting, which may enable trees that have completely different data and different variables (hence the term random). The predictions from the trees, collectively referred to as the “forest,” may be then averaged together to produce the final prediction. Many trees (e.g., one hundred trees) may be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests may be trained in a similar way as decision trees. Specifically, training a random forest may include the following operations: (1) select randomly k features from the total number of features; (2) create a decision tree from these k features using the same operations as for generating a decision tree; and (3) repeat the previous two operations until a target number of trees is created.

### Systems and Devices

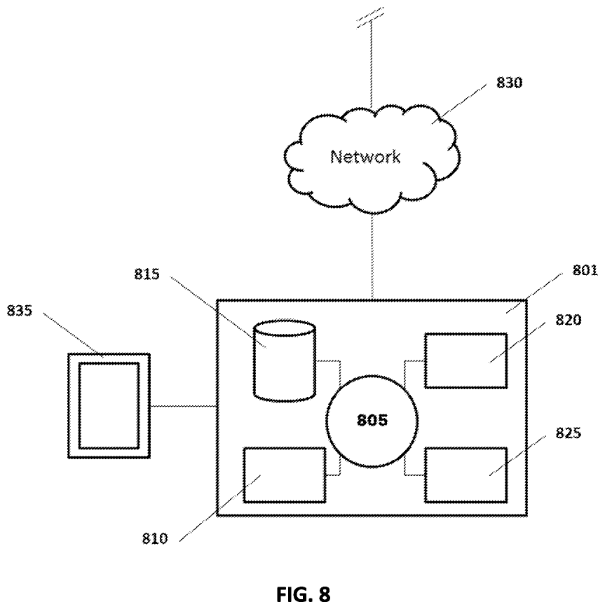

The present disclosure provides computer control devices that are programmed to implement methods of the disclosure. FIG. 8 shows a computer device 801 suitable for use with the software described herein. The computer device 801 can process various aspects of information of the present disclosure, such as, for example, questions and answers, responses, statistical analyses. The computer device 801 can be an electronic device of a user or a computer device that is remotely located with respect to the electronic device. The electronic device can be a mobile electronic device.

The computer device 801 includes a central processing unit (CPU, also “processor” and “computer processor” herein) 805, which can be a single core or multi core processor, or a plurality of processors for parallel processing. The computer device 801 also includes memory or memory location 810 (e.g., random-access memory, read-only memory, flash memory), electronic storage unit 815 (e.g., hard disk), communication interface 820 (e.g., network adapter) for communicating with one or more other devices, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer device 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer device 801, can implement a peer-to-peer network, which may enable devices coupled to the computer device 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the device 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer device 801 in some cases can include one or more additional data storage units that are external to the computer device 801, such as located on a remote server that is in communication with the computer device 801 through an intranet or the Internet.

The computer device 801 can communicate with one or more remote computer devices through the network 830. For instance, the computer device 801 can communicate with a remote computer device of a user (e.g., a parent). Examples of remote computer devices and mobile communication devices include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer device 801 with the network 830.

Methods as described herein can be implemented by way of machine (e.g., computer processor) executable code stored on an electronic storage location of the computer device 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the devices and methods provided herein, such as the computer device 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer device 801 can include or be in communication with an electronic display 835 that comprises a user interface (UI) for providing, for example, questions and answers, analysis results, recommendations. Examples of UI's include, without limitation, a graphical user interface (GUI) and web-based user interface.

Methods and devices of the present disclosure can be implemented by way of one or more algorithms and with instructions provided with one or more processors as disclosed herein. An algorithm can be implemented by way of software upon execution by the central processing unit 805. The algorithm can be, for example, random forest, graphical models, support vector machine or other.

Although the above steps show a method of a device in accordance with an example, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the platform.

Each of the examples as described herein can be combined with one or more other examples. Further, one or more components of one or more examples can be combined with other examples.

### Digital Processing Device

In some embodiments, software as described herein is located on a digital processing device, and/or is configured to cause the processor of the digital processing device to carry out certain tasks. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, a computing device used with the software described herein further includes one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, software as described herein comprises a sequence of instructions, executable by a processor such as a digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Software Modules

In some embodiments, software described herein comprises modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Web Application

In some embodiments, software as described herein comprises a web application. In light of the disclosure provided herein, those of skill in the art will recognize that a web application, in various embodiments, utilizes one or more software frameworks and one or more database systems. In some embodiments, a web application is created upon a software framework such as Microsoft .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, software described herein comprises a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, software described herein comprises a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the software described herein comprises or works in conjunction with a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Databases

In some embodiments, software described herein operates in conjunction with one or more databases, or use of the same. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing based. In other embodiments, a database is based on one or more local computer storage devices.

### EXAMPLES

### Example 1—Artificial Intelligence-Based Medical Device with Improved Detection of Autism in Children

Despite mounting evidence that early intervention for autism spectrum disorder (ASD) improves long-term outcomes and quality of life, significant diagnostic delays persist. User-friendly, data-driven ASD diagnostic tools suitable for deployment in time-pressured care settings can support streamlined diagnosis and help to meet the rapidly rising demand for evaluations that has outpaced specialist capacity.

In response to this need, Example 1 describes an artificial intelligence-based medical device with improved detection of autism in children. Canvas Dx™, an artificial intelligence-based medical device that supports healthcare providers to diagnose or rule out autism in young children with concern for developmental delay, was developed. Canvas Dx™ can be used for autism diagnosis and received FDA marketing authorization in 2021.

Canvas Dx™ used a gradient boosted decision tree machine learning model. The model received behavioral features selected through machine learning techniques as maximally predictive of autism in children 18-72 months of age across a variety of phenotypic presentations to avoid amplification of gender, racial, socio-economic or other demographic biases, and produces a prediction score.

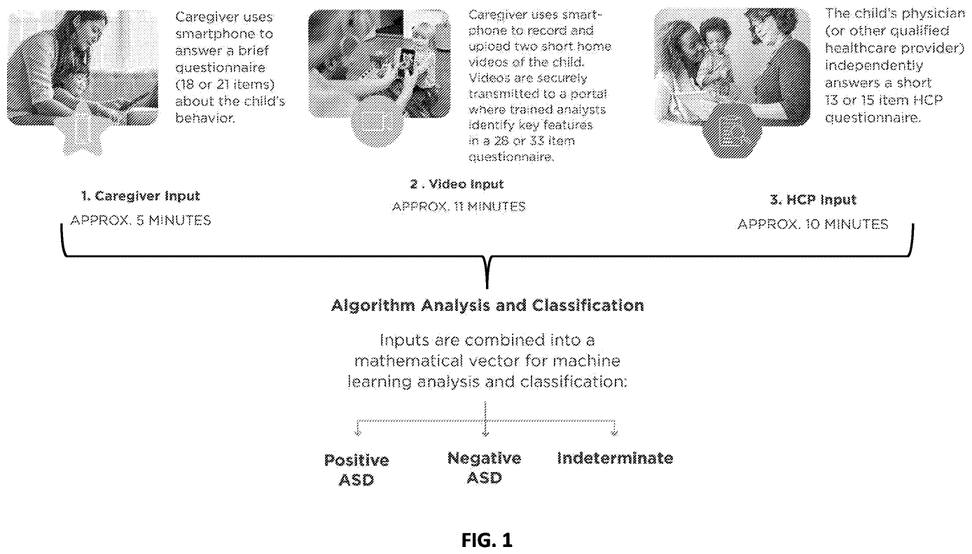

Canvas Dx™ integrates data from multiple sources including input from caregivers and healthcare providers, as well as a structured observation of the child. FIG. 1 shows a workflow of evaluating individuals for the presence of ASD using caregiver input by answering a questionnaire, video input of the individual in home setting, and health care provider input by answering a questionnaire. As illustrated, a caregiver can use a smartphone to answer a brief questionnaire (e.g., 18 or 21 items) about the child's behavior. The caregiver can also record and upload one or more videos and/or audios of the child at home setting. Videos can be securely transmitted to a portal where trained analysts identify key features about the child in a 28 or 33 item questionnaire. A healthcare provider, for example, the child's physician, can independently answer a questionnaire (e.g., 13 or 15 items) regarding the child's behavior and/or health status. One or more of these inputs (e.g., caregiver input, video input, and healthcare provider input) can be used by Canvas Dx™ to generate a predicted evaluation. For example, these inputs can be combined into a vector for machine learning analysis and classification. The classification can include a positive evaluation of ASD, a negative evaluation of ASD, or an indeterminate evaluation. Canvas Dx™ used a gradient boosted decision tree machine learning model that receives behavioral features selected through machine learning techniques as maximally predictive of ASD in children 18-72 months of age across a variety of phenotypic presentations, and produces a prediction score.

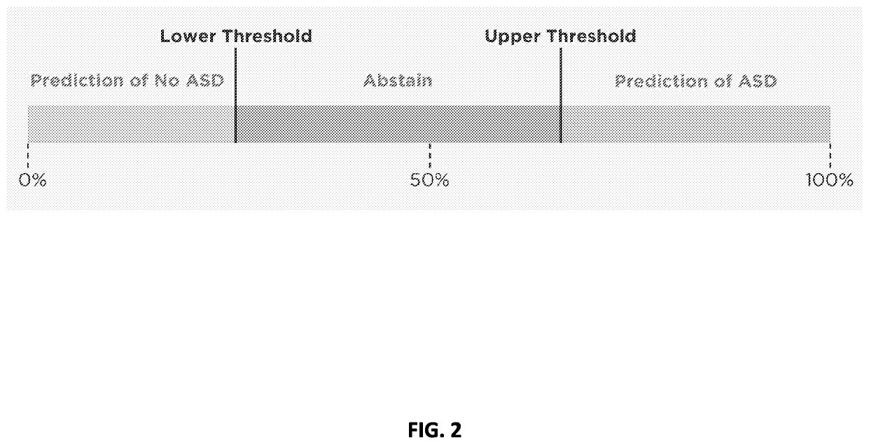

FIG. 2 shows a diagram of lower and upper thresholds delineating the cutoffs for making predictions of absence of ASD and presence of ASD, respectively. Any score between the lower and upper threshold results in an abstention. If the prediction score is above the prespecified threshold, a positive ASD output is produced. If the prediction score is below the prespecified threshold, a negative ASD output is produced. The thresholds were chosen through optimization procedures on training data to enable the device to abstain if presented with insufficient information, a key safety feature that helps ensure device effectiveness. The decision threshold optimization process decreased Canvas Dx™'s abstention rate without changing its intended use. Compared to the version of the algorithm (algorithm V1) that was granted marketing authorization by the FDA, the optimized algorithm (algorithm V2) maintained comparable predictive values.

Decision threshold optimization was conducted under a predetermined change control plan (PCCP) that was part of the device's de novo classification request granted by the FDA. It included both anticipated modifications—software as medical device (SaMD) pre-Specifications—based on the retraining and model update strategy, and the associated methodology—Algorithm Change Protocol—being used to implement those changes in a controlled manner that manages risks to patients following FDA Good Machine Learning Practices. The PCCP covers model tuning and optimization given new data from the intended use population of children ages 18-72 months who have signs/suspicion of risk for developmental delays including autism, based on observations from respective caregivers and/or healthcare providers. Optimization approaches included threshold optimization—specifically the choice of optimal thresholds—that reduced the rate at which the device abstains while maintaining equivalent predictive values.

Two datasets were used for the decision threshold optimization process.

Dataset 1: n=425, ClinicalTrials.gov Identifier NCT04151290 (mean age 3.33; 36.4% female). A reference standard diagnosis was used based on Diagnostic and Statistical Manual of Mental Disorders (DSM-5) criteria corroborated with independent review by specialist clinicians. Specialist clinicians were board certified pediatric psychiatrists, pediatric neurologists, developmental behavioral pediatricians, or psychologists with at least five years of experience diagnosing autism. Specialist clinicians recorded both autism diagnosis and other non-autism developmental delay conditions. Neurotypical development was assumed in cases where no specialist recorded any neurodevelopmental delay condition for the participants. All study participants and assessors were blinded to the results of the device and to the diagnostic determinations of other specialist clinicians. The participants were identified by specialist clinicians as having: 29.0% autism and 71.0% no autism (61.4% of the participants had one or more non-autism developmental delays and 9.6% of the participants had neurotypical development). The participants in this dataset mirrored U.S. population demographics across race, ethnicity, and socio-economic status. The analysis of Dataset 1 is described in “Evaluation of an Artificial Intelligence-based Medical Device for Diagnosis of Autism Spectrum Disorder,” npj Digital Medicine (2022) 5:57, the entirety of which is incorporated by reference herein.

Dataset 2: n=297, (ClinicalTrials.gov Identifier NCT03871179) (mean age 3.97 years; 42.9% female). This is a prospectively collected dataset of children with concern for developmental delay. A reference standard ASD diagnosis was used based on DSM-5 criteria by specialist clinicians. Specialist clinicians recorded both autism diagnoses and other non-autism developmental delay conditions. Neurotypical development was assumed in cases where no specialist recorded any neurodevelopmental delay condition for the participants. All the participants and assessors were blinded to the results of the device. The participants were identified by specialist clinicians as having: 27.6% autism and 72.4% no autism (33.3% of the subjects had one or more non-autism developmental delays and 39.1% of the subjects had neurotypical development).

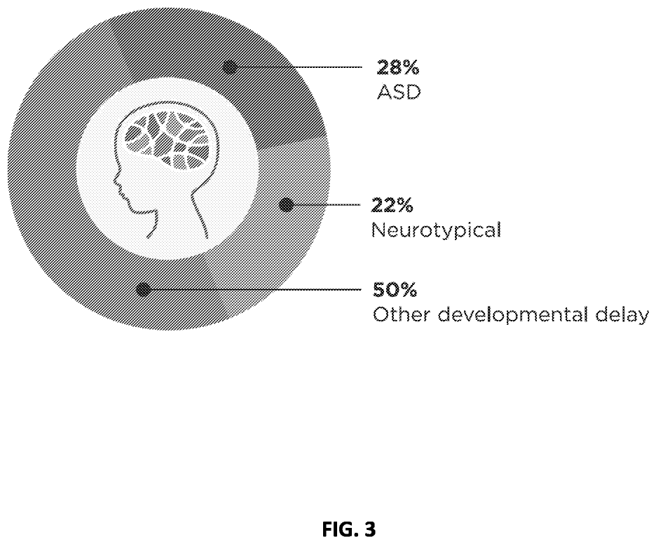

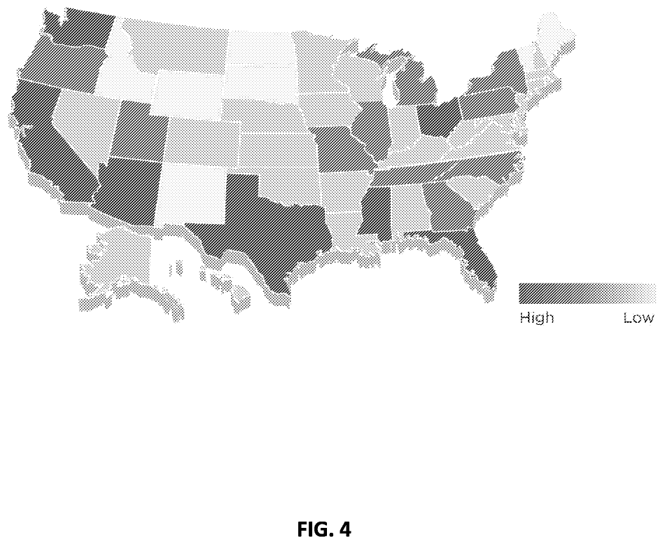

For the purposes of the threshold optimization procedure, Datasets 1 and 2 were combined. The combined dataset contained 722 children meeting the labeling requirements of Canvas Dx™, where the children were aged 18-72 months with concern for developmental delay. The children in the combined dataset were from 43 states of the United States, had a mean age of 3.6 years, and 39% of the children were female. FIG. 3 shows a chart of the neurodevelopmental composition of children in the combined dataset. The neurodevelopmental distribution of the children in the combined dataset was representative of the intended use population with 28% with autism, 50% with one or more non-autism developmental delays, and 22% with neurotypical development, defined as described above through evaluations of specialist clinicians. FIG. 4 shows a map indicating the children from 43 states were represented in the combined dataset.

A repeated train/test validation procedure was used to optimize the algorithm's decision thresholds. In 1000 repeats, from the combined dataset of 722 samples, 70% of the samples randomly were selected for threshold optimization (“training set”) and 30% were for evaluation (“test set”). Each repeat was structured to ensure that no data used in the training were used to test the model. 504 samples in the training sets (mean age=3.6+/−1.2 years; mean 39.1% female) were used for threshold selection and 218 (mean age=3.7+/−1.2 years; mean 39.0% female) for testing. The composition of the training and test sets remained representative of the intended use population.

Threshold optimization was performed on the training set in each repeat by finding an optimal threshold pair that decreased the device's rate of abstention while maximizing positive predictive value (PPV) and negative predictive value (NPV) in the evaluation using the held-out test set. Optimization focused on decreasing the rate of abstention while ensuring predictive values produced using algorithm V2 remained equivalent to those produced using algorithm V1. Out-of-sample performance was estimated by evaluating the selected threshold pair on the test set and comparing the performance metrics of the new pair to the corresponding V1 metrics on the same test set. Performance was determined by examining differences in NPV, PPV and determinate output rate between the two algorithms (V2 V1) and the 95% confidence intervals computed from the quantiles of the repeats.

Out-of-sample performance was estimated by evaluating the selected threshold pair on the test set and comparing the performance metrics of the new pair to the corresponding V1 metrics on the same test set.

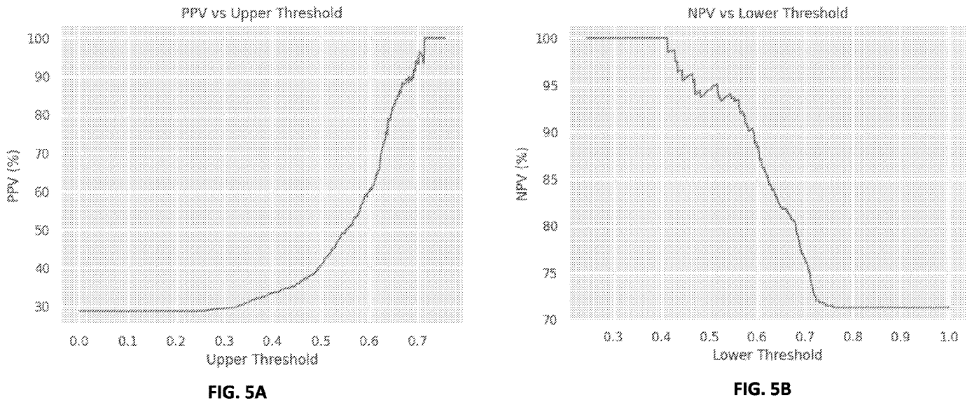

FIG. 5A and FIG. 5B provide an illustration of the effect of the model's threshold hyperparameters on performance metrics. Following the optimization, the algorithm (“algorithm V2”) had a 66.5% determinate rate. NPV and PPV were comparable to those of algorithm V1.

Table 1 lists performance metrics for the device with algorithm V1 and V2. The determinate rate was the percentage of children receiving an autism positive or negative output).

TABLE 1
Performance metrics for the device with algorithm V1 and V2.

| | Positive | Negative | Determinate |
|---|---|---|---|
| | predictive | predictive | rate |
| Device | value (PPV) | value (NPV) | (coverage) |
| Device with algorithm | 87.5 | 95.6 | 66.5 |
| V2 (95% Confidence | (82.5-96.7) | (93.7-97.9) | (62.5-71.0) |
| Interval) | | | |
| Device with algorithm | 89.7 | 96.1 | 45.4 |
| V1 (95% Confidence | (83.9-90.3) | (93.4-98.6) | (41.3-48.6) |
| Interval) | | | |

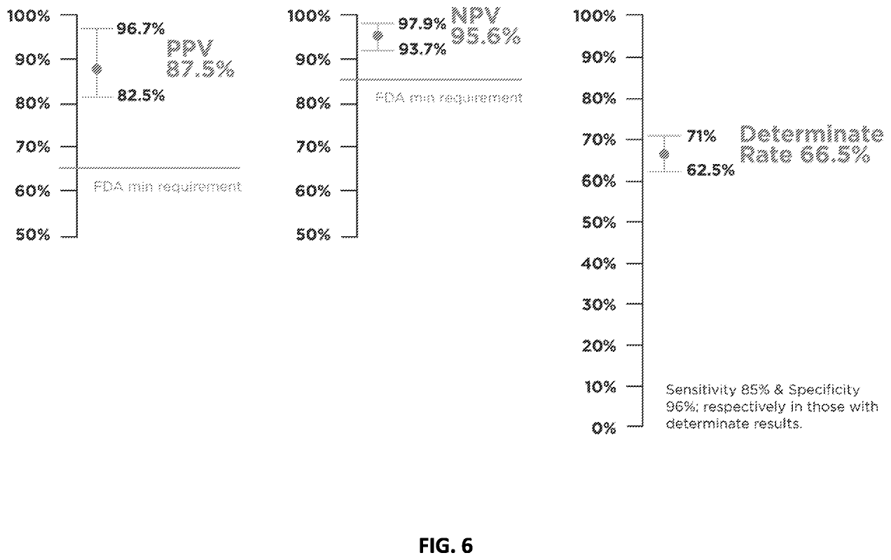

FIG. 6 shows the Canvas DX™ performance metrics (with algorithm V2). Both the positive predictive value (PPV) and negative predictive value (NPV) exceeded the minimum requirements provided by the FDA.

Table 2 shows performance metrics for the device with algorithm V1 and V2 as measured in the combined dataset.

| PPV | NPV | Coverage |
|---|---|---|
| −2.0% (−4.4%, 0.9%) | −0.9% (−3.2%, 1.7%) | 21.2% (17.4%, 25.2%) |

In order to compare algorithm V2 to algorithm V1, the differences in NPV, PPV and determinate output rate between the two algorithms were examined and the 95% confidence intervals computed from the quantiles of the repeats. Confidence intervals overlapping 0 was indicative that there was no significant difference in performance between algorithms V1 and V2. Confidence intervals entirely above 0 was indicative of superior V2 performance. Confidence intervals entirely below 0 was indicative of inferior V2 performance. As shown in Table 1 and Table 2, the device with algorithm V2 produced a significantly higher determinate rate (autism positive or negative) than the device with algorithm V1. The device with algorithm V2 maintained equivalent predictive values to those achieved by the device with Algorithm V1. The determinate output rate of the device with algorithm V1 was 21% greater than the determinate rate of the device with algorithm V2. On average across the repeats, 50 subjects converted from indeterminate to determinate if using the device with algorithm V2. Of these, on average, 4/50 (8%) were falsely predicted as positive (average=1) or negative (average=3).

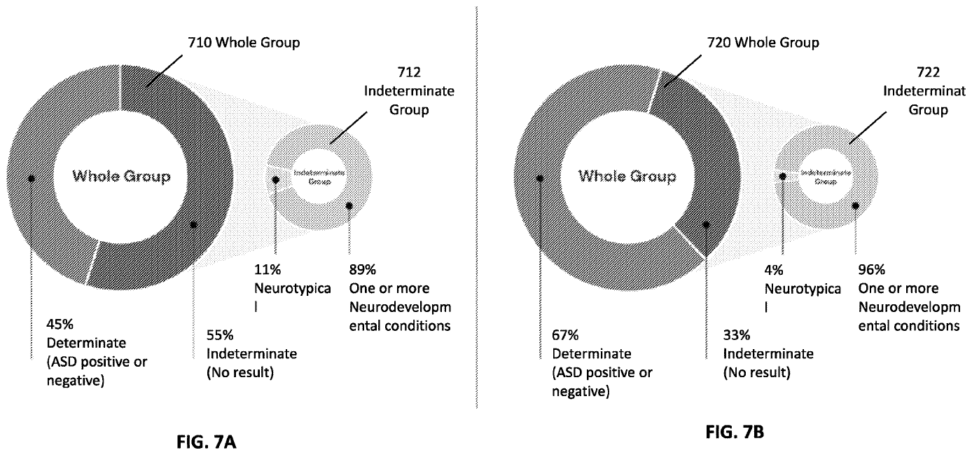

FIGS. 7A and 7B show comparative abstention rates and neurodevelopmental profiles of children within the abstention group across both algorithm versions, based on specialist assessment. FIG. 7A shows abstention rates and neurodevelopmental composition using algorithm V1. FIG. 7B shows abstention rates and neurodevelopmental composition using algorithm V2. If using the optimized algorithm (i.e., algorithm V2), it was observed that children who received indeterminate output were more likely to have one or more neurodevelopmental conditions (including autism), as determined by specialist assessment, compared to when using the device with algorithm V1. Specifically, children who received an indeterminate output from the device with algorithm V2 were 3.4 times less likely to be neurotypically developing than those who received an indeterminate output from the device with algorithm V1 (V1: 8:1 odds of being neurotypical, vs V2: 28:1 odds of being neurotypical).

In a prospective, multi-site, double-blinded clinical validation study, Canvas Dx™ output was also compared to consensus specialist diagnosis. Device output PPV for all study completers was 80.8% (70.3%-88.8%) and NPV was 98.3% (90.6%-100%). Approximately one third (31.8%) of participants received a determinate output (ASD positive or negative). The remaining 68% of participants received an indeterminate (predictive abstention) output.

The algorithmic threshold modification procedure described herein resulted in a higher determinate rate with no degradation of negative and positive predictive values. The vast majority (92%) of determinate outputs received under algorithm V2 for subjects classified indeterminate under algorithm V1 were correctly aligned with specialist diagnosis. This finding highlights the potential of the optimized device to support healthcare providers to accurately detect or rule out autism in more young children in clinical practice. Furthermore, if a previously indeterminate patient is reclassified with future algorithmic updates to a determinate output, the determinate output is likely to be correct. The modification does not affect how the device is used in practice, impact the directions for use, or introduce any new risks or significantly modify existing risks.

The flexible machine learning design approach adopted in this threshold optimization procedure allowed leveraging training data that mirrored U.S. population demographics across race, ethnicity, and socio-economic status. All children in the combined dataset fell within the intended use population (concern for developmental delay, aged 18-72 months). Per FDA recommendations, Good Machine Learning Practices were followed throughout the study design and execution including separation between training and test sets in each repeat and use of best practice data management and handling practices. Additional real world data would help clarify the extent to which autism prevalence, and other assumptions built into our model, are reflective of the real-world usage population. Planned and ongoing real world evidence studies may also shed light on how the device can be integrated into primary care practice settings, and how its use may impact time to diagnosis and treatment initiation.

The above are examples of the practical use of a predetermined change control plan under the FDA's proposed regulatory framework. Regulatory mechanisms such as the PCCP may come to play an important role in product development as artificial intelligence or machine learning-based technologies grow at a rapid pace. Non-adaptive artificial intelligence or machine learning-based algorithms may have the risk of becoming dated and degraded over time if data used for training no longer reflects the real-world circumstances that they are being applied to.

The systems and method as described herein allow manufacturers to intermittently “unlock” algorithms to expose their models to new data, thereby preventing algorithmic drift if clinically deployed. The modified decision thresholds significantly reduced the device's abstention rate while maintaining predictive values that are comparable to those achieved by the device with algorithm V1. The device, with optimized decision thresholds, may support providers to efficiently evaluate more children with concern for developmental delay, including diagnosing or ruling out ASD. Applying data driven approaches addressed some of the inherent limitations of current ASD diagnostic approaches. Enhanced diagnostic capacity can allow a greater number of children to access ASD services during the critical early years of high neuroplasticity where interventions have the greatest impact. Additional details are described in “Optimizing a de novo artificial intelligence-based medical device under a predetermined change control plan: Improved ability to detect or rule out pediatric autism,” Intelligence-Based Medicine 8 (2023) 100102, the entirety of which is incorporated by reference herein.

In conclusion, implementation of an algorithm optimization process significantly reduced the device's abstention rate while maintaining equivalent predictive values to those observed with algorithm V1. The device, with optimized decision thresholds, has potential to support healthcare providers to diagnose or rule out autism in a greater proportion of young children with concern for developmental delay.

What is claimed is:

1. A computer-implemented method comprising:
(a) receiving input data of an individual related to a behavioral disorder, neurological impairment, or developmental delay;
(b) evaluating the input data using an evaluation module comprising at least one machine learning model, thereby generating an evaluation result, wherein the at least one machine learning model comprises one or more decision threshold hyperparameters that differentiate between a positive evaluation, negative evaluation, and an indeterminate evaluation with respect to a presence or an absence of the behavioral disorder, neurological impairment, or developmental delay; and
(c) generating a personal therapeutic treatment plan for the individual based at least in part on the evaluation result if the evaluation result comprises the presence of the behavioral disorder, neurological impairment, or developmental delay.

2. The computer-implemented method of claim 1, wherein the at least one machine learning model comprises the one or more decision threshold hyperparameters provides a positive predictive value of at least about 80%, a negative predictive value of at least about 95%, a coverage or inclusion rate of at least about 70%, or any combination thereof when evaluated using a nested cross-validation procedure.

3. The computer-implemented method of claim 1 or 2, wherein the one or more decision threshold hyperparameters are generated using an automated cross-validation procedure.

4. The computer-implemented method of any one of claims 1-3, wherein the one or more decision threshold hyperparameters define a threshold range for determining if an evaluation result is a positive evaluation, a negative evaluation, or an indeterminate evaluation.

5. The computer-implemented method of any one of claims 1-4, wherein a first categorical determination for the presence or absence of the behavioral disorder, neurological impairment, or developmental delay in the individual is based on a specified sensitivity, a specified specificity, a specified negative predictive value, or a specified positive predictive value.

6. The computer-implemented method of any one of claims 1-5, wherein the at least one machine learning model comprises a subset of a plurality of tunable machine learning models.

7. The computer-implemented method of any one of claims 1-6, further comprising:
(a) requesting additional data if the evaluation result comprises the indeterminate evaluation; and
(b) generating an updated evaluation result based on the additional data using the evaluation module.

8. The computer-implemented method of any one of claims 1-7, further comprising training the at least one machine learning model with a first training dataset, a second training dataset, and a third training dataset, wherein the first training dataset comprises one or more video recordings of individuals, the second training dataset comprises one or more feedbacks provided by healthcare providers to a questionnaire, and the third training dataset comprises one or more feedbacks provided by caregivers to a questionnaire.

9. The computer-implemented method of claim 8, wherein at least one of the first training dataset, the second training dataset, or the third training dataset comprises at least 100 training samples.

10. The computer-implemented method of any one of claims 1-9, wherein the behavioral disorder, neurological impairment, or developmental delay comprises pervasive development disorder (PDD), autism spectrum disorder (ASD), social communication disorder, restricted repetitive behaviors, interests, and activities (RRBs), autism (“classical autism”), Asperger's Syndrome (“high functioning autism), PDD-not otherwise specified (PDD-NOS, “atypical autism”), attention deficit disorder (ADD), attention deficit and hyperactivity disorder (ADHD), speech and language delay, obsessive compulsive disorder (OCD), depression, schizophrenia, Alzheimer's disease, dementia, intellectual disability, or learning disability.

11. The computer-implemented method of any one of claims 1-10, wherein the behavioral disorder, neurological impairment, or developmental delay is autism spectrum disorder or autism.

12. The computer-implemented method of any one of claims 1-11, further comprising administering the personal therapeutic treatment plan for the individual via a healthcare provider or a caregiver of the individual.

13. The computer-implemented method of claim 12, wherein the personal therapeutic treatment plan is generated using a therapeutic module comprising at least one statistical or machine learning model.

14. The computer-implemented method of claim 12 or 13, further comprising receiving feedback data based on performance of the personal therapeutic treatment plan and updating the personal therapeutic treatment plan based on the feedback data.

15. The computer-implemented method of claim 14, wherein the feedback data comprises at least one of efficacy, compliance, or response to the personal therapeutic treatment plan.

16. The computer-implemented method of any one of claims 12-15, wherein the personal therapeutic treatment plan comprises a drug therapy, a non-drug therapy, or both.

17. The computer-implemented method of claim 16, wherein the non-drug therapy comprises digital therapeutics.

18. The computer-implemented method of any one of claims 1-17, wherein the at least one machine learning model comprises a gradient boosted classifier model.

19. A computer-implemented method comprising:
(a) receive input data comprising a plurality of features related to a behavioral disorder, neurological impairment, or developmental delay;
(b) divide the input data into training data sets and testing data sets;
(c) training a model using one of the training data sets;
(d) determine at least one decision thresholds for the model using a corresponding testing data set;
(e) repeat steps (b)-(d) at least once using a cross-validation procedure to generate a plurality of decision thresholds;
(f) determine one or more decision threshold hyperparameters using the plurality of decision thresholds; and
(g) training a final model using the input data, wherein the final model comprises the one or more decision threshold hyperparameters.

20. The computer-implemented method of claim 19, wherein the final model is configured to:
(h) receive input data of an individual related to the behavioral disorder, neurological impairment, or developmental delay;
(i) evaluate the input data of the individual and generate an evaluation result, wherein the one or more decision threshold hyperparameters differentiate between a positive evaluation, negative evaluation, and an indeterminate evaluation with respect to a presence or an absence of the behavioral disorder, neurological impairment, or developmental delay; and
(j) generate a personal therapeutic treatment plan for the individual based at least in part on the evaluation result if the evaluation result comprises the presence of the behavioral disorder, neurological impairment, or developmental delay.

21. A computer-implemented method comprising:
(a) receiving input data comprising a plurality of features related to a behavioral disorder, neurological impairment, or developmental delay;
(b) dividing the input data into tuning data sets and validation data sets;
(c) dividing the tuning data sets into training data sets and testing data sets;
(d) training a model using one of the training data sets;
(e) determining at least one decision thresholds for the model using a corresponding testing data set;
(f) repeating steps (c)-(e) at least once using a cross-validation procedure to generate a plurality of decision thresholds;
(g) training a new model using one of the tuning data sets;
(h) evaluating the new model using a corresponding validation data set according to at least one decision threshold based on the plurality of decision thresholds;
(i) repeating steps (a)-(h) at least once using a cross-validation procedure to calculate one or more performance metrics;
(j) determine one or more decision threshold hyperparameters using the plurality of decision thresholds; and
(k) training a final model using the input data, wherein the final model comprises the one or more decision threshold hyperparameters.

22. The computer-implemented method of claim 21, wherein the final model is configured to:
(l) receive input data of an individual related to the behavioral disorder, neurological impairment, or developmental delay;
(m) evaluate the input data of the individual and generate an evaluation result, wherein the one or more decision threshold hyperparameters differentiate between a positive evaluation, negative evaluation, and an indeterminate evaluation with respect to a presence or an absence of the behavioral disorder, neurological impairment, or developmental delay; and
(n) generate a personal therapeutic treatment plan for the individual based at least in part on the evaluation result if the evaluation result comprises the presence of the behavioral disorder, neurological impairment, or developmental delay.

23. A system comprising: a processor; a non-transitory computer readable medium including executable instructions configured to cause the processor to perform computer-implemented method of any one of claims 1-22.

24. A non-transitory computer readable medium including executable instructions configured to cause a processor to perform computer-implemented method of any one of claims 1-22.
